(19) United States
(12) Patent Application Publication
(10) Pub. No.: US 2008/0194669 A1
(43) Pub. Date: Aug. 14, 2008

(54) T Type Calcium Channel Blockers and the Treatment of Diseases

(76) Inventors: Lloyd S. Gray, Charlottesville, VA (US); Doris M. Haverstick, Charlottesville, V (US)

Correspondence Address: SCULLY SCOTT MURPHY & PRESSER, PC, 400 GARDEN CITY PLAZA, SUITE 300, GARDEN CITY, NY, 11530, US

(21) Appl. No.: 11/660,401

(22) PCT Filed: Aug. 22, 2005
(86) PCT No.: PCT/US2005/029851

§ 371 Date: Sep. 11, 2007

Related U.S. Application Data
(60) Provisional application No. 60/603,159, filed on Aug. 20, 2004.

(51) Int. Cl. A61K 31/40; C12N 5/06; A61P 35/00

(52) U.S. Cl. 514/428; 435/375

(57) ABSTRACT

The present invention provides a method for treating a disease or condition in a mammal associated with influx of extracellular calcium via T type calcium channels, which comprises administering to the mammal a therapeutically effective amount of a T type calcium channel inhibitor, a prodrug thereof, or a pharmaceutically acceptable salt of said inhibitor or prodrug, wherein the T type calcium channel inhibitor has a structure represented by Formula (I): Formula (I), wherein R₁ is selected from the group consisting of C₁-C₄ alkyl, hydroxy and C₁-C₄ alkoxy; X is selected from the group consisting of N and CH; Z is selected from the group consisting of NH, O, S and CH₂; R₂ is selected from the group consisting of H, halo, NH₂, C₁-C₄ alkyl, hydroxy and C₁-C₄ alkoxy; and R₃ is selected from the group consisting of H, halo, NH₂, C₁-C₄ alkyl, hydroxy and C₁-C₄ alkoxy. In one embodiment R₁ is selected from the group consisting of C₁-C₄ alkyl, hydroxyl and C₁-C₄ alkoxy, X is N, Z is O or CH₂, R₂ is H, halo, NH₂ or hydroxyl and R₃ is H.

### BACKGROUND OF THE INVENTION

Influx of extracellular calcium is critical for a number of vital cellular processes. Calcium influx is generally mediated by calcium channels, which are grouped into several families one of which is the T type calcium channel family. Pharmacological modulation of the T type calcium channel's function is tremendously important in the practice of medicine; for example, T type calcium channel inhibitors are in widespread use in the treatment of neurological diseases (e.g. epilepsy, petit mal seizure, absence seizure, neuropathic pain, and etc.) and cardiovascular diseases (e.g. hypertension, unstable angina, and etc.). For example, mibefradil, a T type calcium inhibitor, was clinically efficacious in treating hypertension and cardiac arrhythmia. Studies also suggest that T-type calcium channels may play an important role in age related macular degeneration. Recently, we have showed that the α1H and δ25 isoforms of T type calcium channels are present in cancer cell lines and that novel chemical agents could be synthesized to block calcium entry via this channel thus inhibiting cancer cell proliferation.

It has been known for some time that Ca2+ entry is a critical regulatory component of the cell cycle and such that inhibition of it blocks proliferation. However, the mechanism by which Ca2+ entry occurs continues to be a matter of some debate. The known sensitivity of the Ca2+ entry pathway in electrically non-excitable cells to Ni2+ inhibition was used to develop a strategy enabling the directed chemical synthesis of novel compounds with significant potency for inhibiting Ca2+ entry into and, consequently, proliferation of several cancer cell lines. These novel compounds show stereoselective inhibition of Ca2+ influx and proliferation. Similar stereoselective inhibition of the Ca2+ current through the α1H isoform of T type Ca2+ channels was also observed. The compounds of the present invention block Ca2+ current through heterologously expressed α1H Ca2+ channels. Additionally, the sensitive cancer cell lines expressed message for either the α1H T type Ca2+ channel isoform or its 625 splice variant. These observations suggest that clinically useful drugs can be designed based upon the ability to interact with these Ca2+ channels as described herein thought the use of a xenograft model of human cancer.

Voltage gated calcium require rapid changes in membrane potential, called action potentials, for activation of calcium influx. Electrically non-excitable cells, such as the majority of cancer cell types, do not have action potentials. Consequently, voltage gated calcium channels are thought to have no role in electrically non-excitable cells (Venkatachalam, K., Van Rossum, D. B., Patterson, R. L., Ma, H.-T., and Gill, D. L. 2002. The cellular and molecular basis of store-operated calcium entry. Nat. Cell BioI. 4:E263-E272). However, calcium influx is required for proliferation of electrically non-excitable cancer cells such that inhibition of calcium influx produces cytostasis (Berridge, M. J., Lipp, P., and Bootman, M. D. 2000. The versatility and universality of calcium signalling. Nat. Rev. Mol Cell BioI. 1: 11-21). Consequently, knowledge of the mechanism of calcium entry in electrically non-excitable cells would be useful in cytostatically halting or slowing the proliferation of cancer cells.

In accordance with the present inhibitors of voltage gated T type calcium channels are provided that block proliferation of electrically non-excitable cancer cells in vitro. In addition, T channel blocking agents inhibit cancer cell proliferation in a xenograft model of human cancer (Haverstick, D. M., Heady, T. N., Macdonald, T. L., and Gray, L. S. 2000. Inhibition of human prostate cancer proliferation in vitro and in a mouse model by a compound synthesized to block Ca2+ entry. Cancer Res 60:1002-1008). This same compound blocks calcium entry through the heterologously expressed α1H T type calcium channel isoform. This blockade of calcium influx is specific because chiral compounds have the same rank potency at blocking calcium through heterologously expressed α1H calcium as they do in inhibiting proliferation of electrically non-excitable cancer cell lines.

In summary, the present invention relates to the identification of the α1H isoform of T type calcium channels and its 625 splice variant and their use to modulate proliferation of electrically non-excitable cancer cell types with the intent of treating cancer by inhibition of proliferation.

### SUMMARY OF THE INVENTION

Accordingly, the present invention provides a method for treating a disease or condition in a mammal associated with influx of extracellular calcium via T type calcium channels, which comprises administering to the mammal a therapeutically effective amount of a T type calcium channel inhibitor, a prodrug thereof, or a pharmaceutically acceptable salt of said inhibitor or prodrug. Preferably, the disease or condition is selected from the group consisting of unstable angina, hypertension, epilepsy, neuropathic pain, petit mal seizure, absence seizure, age related macular degeneration, cancer, and pre-cancerous condition.

The present invention also provides a method for treating a disease or condition in a mammal associated with influx of extracellular calcium via T type calcium channels, which comprises administering to the mammal a therapeutically effective amount of a T type calcium channel inhibitor, a prodrug thereof, or a pharmaceutically acceptable salt of said inhibitor or prodrug, wherein the T type calcium channel inhibitor blocks an α1H isoform of T type calcium channels or a δ25 splice variant thereof.

Preferably, the above-mentioned T type calcium channel inhibitor has a structure represented by Formula (I):

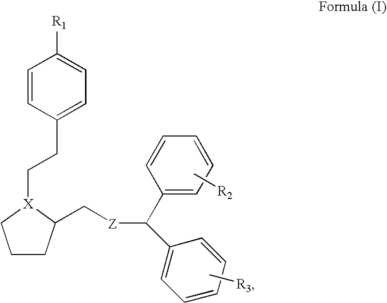

wherein

R₁ is selected from the group consisting of C₁-C₄ alkyl, hydroxy and C₁-C₄ alkoxy;

X is selected from the group consisting of N and CH;

Z is selected from the group consisting of NH, O, S and CH₂;

R₂ is selected from the group consisting of H, halo, NH₂, C₁-C₄ alkyl, hydroxy and C₁-C₄ alkoxy; and

R₃ is selected from the group consisting of H, halo, NH₂, C₁-C₄ alkyl, hydroxy and C₁-C₄ alkoxy. In one embodiment R₁ is selected from the group consisting of C₁-C₄ alkyl, hydroxy and C₁-C₄ alkoxy, X is N, Z is O or CH₂, R₂ is H, halo, NH₂ or hydroxy and R₃ is H.

In another aspect, the present invention provides a method for reducing proliferation of electrically non-excitable cells, which comprises administering a T type calcium channel inhibitor, wherein said T type calcium channels inhibitor blocks an α1H isoform of T type calcium channels or a δ25 splice variant thereof.

In another aspect, the present invention provides a method for inhibiting calcium entry into electrically non-excitable cells, which comprises administering a T type calcium channel inhibitor, wherein said T type calcium channels inhibitor blocks an α1H isoform of T type calcium channels or a δ25 splice variant thereof.

In another aspect, the present invention provides a A pharmaceutical composition comprising a therapeutically effective amount of a compound of formula (I) as described above, a prodrug of said compound or a pharmaceutically acceptable salt of said compound or prodrug; and a pharmaceutically acceptable carrier, vehicle or diluent.

In another aspect, the present invention provides a method for the treatment of cancer or pre-cancerous condition in a mammal, which comprises administering to the mammal a therapeutically effective amount of a compound of formula (I) as described above, a prodrug thereof, or a pharmaceutically acceptable salt of said compound or prodrug in combination with one or more anti-tumor agent.

In another aspect, the present invention provides a pharmaceutical combination composition comprising a therapeutically effective amount of a combination of a compound of formula (I) as described above, a prodrug thereof, or a pharmaceutically acceptable salt of said compound or prodrug; and one or more anti-tumor agent.

### BRIEF DESCRIPTION OF THE DRAWINGS

FIG. 1. Identification of known compounds that block Ca2+ entry and proliferation and design of novel compounds with increased potency. Proliferation and Ca2+ entry were determined in Jurkat cancer cells as described below in the Materials and Methods. Individual concentration-response curves for each activity and compound were constructed and IC50 values were calculated. The calculated least squares regression in shown as a solid line and a line with a slope of one in shown as a dashed line. Panel A: Using Ni2+ sensitivity as a guide, known compounds were identified that block Ca2+ entry and proliferation in Jurkat cells. Panel B: An SAR was developed from the data depicted in panel A leading to the synthesis of novel compounds that block Ca2+ entry and proliferation Jurkat cells.

FIG. 2. The Ca2+ ionophore ionomycin overcomes the inhibition of Ca2+ entry into and proliferation of Jurkat cells produced by TH-1177. Panel A. Ca2+ entry into Jurkat cells was determined as described in Materials and Methods. Ionomycin at the indicated concentrations was added 30 s and the mitogenic monoclonal antibody OKT3 was added at a concentration of 1 ug/ml at 60 s. Either EGTA at 2.5 mM or TH-1177 at the indicated concentrations was added at 150 s. The percentage inhibition was determined as described in Material and Methods. Panel B. Jurkat cells were grown for 48 hrs in the presence (open symbols) or absence (closed symbols) of 30 nM ionomycin and the indicated concentrations of TH-1177. Percent control growth was determined as described in Materials and Methods.

FIG. 3. Amplicons of two sizes were identified in Jurkat and SK-N-SF cancer cell lines using α1H Ca2+ channel specific PCR primers. Messenger RNA was extracted and amplified as described in Materials and Methods using the primers described previously (Mariot, P., Vanoverberghe, K., Lalevee, N., Rossier, M. F., and Prevarskaya, N. 2002. Overexpression of an alpha 1H (Cav3.2) T-type calcium channel during neuroendocrine differentiation of human prostate cancer cells. J.Biol.Chem 277:10824-10833). The resulting products were isolated by gel electrophoresis and visualized by ethidium bromide staining and visualized by UV illumination.

FIG. 4. The sequences of the amplicons shown in FIG. 4 are virtually identical to either α1H or its 825 splice variant. The amplicons shown in FIG. 4 were sequenced as described in Materials and Methods. The GenBank database was then queried and the alignments shown were obtained.

FIG. 5. TH-1177 and TH-1211 are stereoisomers about one of two chiral centers and have different potencies at inhibiting the proliferation of PC3 prostate cancer cells. Panel A: The structures of TH-1177 and TH-1211 were determined as described in Materials and Methods. The diastereomers are racemic at the benzhydrol center (solid arrows) and enantiomeric at the proline center (open arrows) with TH-1177 having the S configuration and TH-1211 having the R. Panel B: The proliferation of PC3 human prostate cancer cells was determined as described in Materials and Methods. The IC50 for TH-1177 was 14 uM (open boxes) and for TH-1211 was 42 uM (inverted triangles).

FIG. 6. TH-1177 and TH-1211 have different potencies at inhibiting the Ca2+ current through transfected α1H channels. The current carried by transfected α1H Ca2+ channels was determined as described in Materials and Methods. The concentration response for TH-1177 and TH-1211 is shown in FIG. 6D. The IC50 for TH-1177 was 0.8 uM and for TH-1211 was 7 uM.

### DEFINITIONS

In describing and claiming the invention, the following terminology will be used in accordance with the definitions set forth below.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure.

As used herein, the term “treating” includes administering therapy to prevent, cure, or alleviate/prevent the symptoms associated with, a specific disorder, disease, injury or condition. For example treating cancer includes inhibition or complete growth arrest of a tumor, reduction in the number of tumor cells, reduction in tumor size, inhibition of tumor cell infiltration into peripheral organs/tissues, inhibition of metastasis as well as relief, to some extent, of one or more symptoms associated with the disorder. The treatment of cancer also includes the administration of a therapeutic agent that directly decreases the pathology of tumor cells, or renders the tumor cells more susceptible to treatment by other therapeutic agents, e.g., radiation and/or chemotherapy. As used herein, the term “treating” includes prophylaxis of the specific disorder or condition, or alleviation of the symptoms associated with a specific disorder or condition and/or preventing or eliminating said symptoms.

As used herein, the term “pharmaceutically acceptable carrier, vehicle or diluent” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

The term “therapeutically effective amount” means an amount of a compound of the present invention that ameliorates, attenuates or eliminates a particular disease or condition or prevents or delays the onset of a particular disease or condition.

By “mammal” it is meant to refer to all mammals, including, for example, primates such as humans and monkeys. Examples of other mammals included herein are rabbits, dogs, cats, cattle, goats, sheep and horses. Preferably, the mammal is a female or male human.

The phrase “compound(s) of the present invention” or “compound(s) of Formula (I)” or the like, shall at all times be understood to include all active forms of such compounds, including, for example, the free form thereof, e.g., the free acid or base form, and also, all prodrugs, polymorphs, hydrates, solvates, tautomers, and the like, and all pharmaceutically acceptable salts, unless specifically stated otherwise. It will also be appreciated that suitable active metabolites of such compounds are within the scope of the present invention.

The expression “prodrug” refers to compounds that are drug precursors which following administration, release the drug in vivo via some chemical or physiological process (e.g., a prodrug on being brought to the physiological pH or through enzyme action is converted to the desired drug form).

The expression “pre-cancerous condition” refers to a growth that is not malignant but is likely to become so if not treated. A “pre-cancerous condition” is also known as “pre-malignant condition” by one of ordinary skill in the art.

As used herein the term “anti-tumor agent” relates to agents known in the art that have been demonstrated to have utility for treating neoplastic disease. For example, antitumor agents include, but are not limited to, antibodies, toxins, chemotherapeutics, enzymes, cytokines, radionuclides, photodynamic agents, and angiogenesis inhibitors. Toxins include ricin A chain, mutant Pseudomonas exotoxins, diphtheria toxoid, streptonigrin, boamycin, saporin, gelonin, and pokeweed antiviral protein. Chemotherapeutics include 5-fluorouracil (5-FU), daunorubicin, cisplatinum, bleomycin, melphalan, taxol, tamoxifen, mitomycin-C, and methotrexate as well as any of the compounds described in U.S. Pat. No. 6,372,719 (the disclosure of which is incorporated herein by reference) as being chemotherapeutic agents. Radionuclides include radiometals. Photodynamic agents include porphyrins and their derivatives. Angiogenesis inhibitors are known in the art and include natural and synthetic biomolecules such as paclitaxel, O-(chloroacetyl-carbornyl) fumagillol (“TNP-470” or “AGM 1470”), thrombospondin-1, thrombospondin-2, angiostatin, human chondrocyte-derived inhibitor of angiogenesis (“hCHLkMP”), cartilage-derived angiogenic inhibitor, platelet factor-4, gro-beta, human interferon-inducible protein 10 (“IP10”), interleukin 12, Ro 318220, tricyclodecan-9-yl xanthate (“D609”), irsogladine, 8,9-dihydroxy-7-methyl-benzo[b]quinolizinium bromide (“GPA 1734”), medroxyprogesterone, a combination of heparin and cortisone, glucosidase inhibitors, genistein, thalidomide, diamino-antraquinone, herbimycin, ursolic acid, and oleanolic acid. Anti-tumor therapy includes the administration of an anti-tumor agent or other therapy, such as radiation treatments, that has been reported as being useful for treating cancer.

As used herein, the term “halogen” or “halo” includes bromo, chloro, fluoro, and iodo.

The term “haloalkyl” as used herein refers to an alkyl radical bearing at least one halogen substituent, for example, chloromethyl, fluoroethyl or trifluoromethyl and the like.

The term “C₁-Cₙ alkyl” wherein n is an integer, as used herein, represents a branched or linear alkyl group having from one to the specified number of carbon atoms. Typically C₁-C₆ alkyl groups include, but are not limited to, methyl, ethyl, n-propyl, iso-propyl, butyl, iso-butyl, sec-butyl, tert-butyl, pentyl, hexyl and the like.

The term “C₂-Cₙ alkenyl” wherein n is an integer, as used herein, represents an olefinically unsaturated branched or linear group having from 2 to the specified number of carbon atoms and at least one double bond. Examples of such groups include, but are not limited to, 1-propenyl, 2-propenyl, 1,3-butadienyl, 1-butenyl, hexenyl, pentenyl, and the like.

The term “C₂-Cₙ alkynyl” wherein n is an integer refers to an unsaturated branched or linear group having from 2 to the specified number of carbon atoms and at least one triple bond. Examples of such groups include, but are not limited to, 1-propynyl, 2-propynyl, 1-butynyl, 2-butynyl, 1-pentynyl, and the like.

The term “C₃-Cₙ cycloalkyl” wherein n=4-8, represents cyclopropyl, cyclobutyl, cyclopentyl, cyclohexyl, cycloheptyl, and cyclooctyl.

As used herein, the term “optionally substituted” refers to from zero to four substituents, wherein the substituents are each independently selected. Each of the independently selected substituents may be the same or different than other substituents.

As used herein the term “aryl” refers to a mono or bicyclic carbocyclic ring system having one or two aromatic rings including, but not limited to, phenyl, benzyl, naphthyl, tetrahydronaphthyl, indanyl, indenyl, and the like.

The term “heterocyclic group” refers to a mono or bicyclic carbocyclic ring system containing from one to three heteroatoms wherein the heteroatoms are selected from the group consisting of oxygen, sulfur, and nitrogen.

The term “pharmaceutically acceptable salt” refers to salts which retain the biological effectiveness and properties of the compounds of the present invention and which are not biologically or otherwise undesirable. In many cases, the compounds of the present invention are capable of forming acid and/or base salts by virtue of the presence of amino and/or carboxyl groups or groups similar thereto.

It is understood to one skilled in the art that “T type calcium channel inhibitors” are also known as “T type calcium channel inhibitors”.

### DETAILED DESCRIPTION OF THE INVENTION

Recently, progress has been made in identifying candidate channels for mediating Ca+ entry (Li, S. W., Westwick, J., and Poll, C. T. 2002. Receptor-operated Ca2+ influx channels in leukocytes: a therapeutic target? Trends Pharmacol.Sci. 23:63-70; Mori, Y., Wakamori, M., Miyakawa, T., Hermosura, M., Hara, Y., Nishida, M., Hirose, K., Mizushitna, A., Kurosaki, M., Mori, E. et al. 2002. Transient receptor potential 1 regulates capacitative Ca²⁺ entry and Ca²⁺ release from endoplasmic reticulum in B lymphocytes. J. Exp.Med. 195:673-681; Tsavaler, L., Shapero, M. H., Morkowski, S., and Laus, R. 2001. Trp-p8, a novel prostate-specific gene, is up-regulated in prostate cancer and other malignancies and shares high homology with transient receptor potential calcium channel proteins. Cancer Res. 61:3760-3769; Peng, J. B., Zhuang, L., Berger, U. V., Adam, R. M., Williams, B. J., Brown, E. M., Hediger, M. A., and Freeman, M. R. 2001. CaT1 expression correlates with tumor grade in prostate cancer. Biochem.Biophys.Res. Commun. 282:729-734; Benham, C. D., Davis, J. B., and Randall, A. D. 2002. Vanilloid and TRP channels: a family of lipid-gated cation channels. Neuropharmacology 42:873-888). However, these Ca2+ channels do not completely fulfill the criteria for the Ca2+ entry pathway in electrically non-excitable cells (Clapham, D. E. 2002. Sorting out MIC, TRP, and CRAC Ion Channels. J.Gen.Physiol 120:217-220; Densmore, J. J., Haverstick, D. M., Szabo, G., and Gray, L. S. 1996. A voltage operable current is involved in activation-induced Ca²⁺ entry in human lymphocytes whereas I_{CRAC} has no apparent role. Am.J.Physiol. 271:C1494-C1503). This lack of knowledge may be the result of a number of factors. While several of the candidates for mediation of Ca2+ entry in electrically non-excitable cells have been characterized at the molecular level (Putney, J. W., Jr. and McKay, R. R. 1999. Capacitative calcium entry channels. Bioessays 21:38-46), a commonly accepted candidate, ICRAC (Clapham, D. E. 2002. Sorting out MIC, TRP, and CRAC Ion Channels. J.Gen.Physiol 120:217-220; Cahalan, M. D., Wulff, H., and Chandy, K. G. 2001. Molecular properties and physiological roles of ion channels in the immune system. J Clin Immunol. 21:235-252), has not been although it was first identified about ten years ago (Hoth, M. and Penner, R. 1993. Calcium release-activated calcium current in rat mast cells. J. Physiol. 465:359-386; Hoth, M. and Penner, R. 1992. Depletion of intracellular calcium stores activates a calcium current in mast cells. Nature 355:353-356). It is also difficult to tie the function of these channels to inhibition of proliferation of cancer cell lines because of the lack of specific Ca2+ entry blockers for electrically non-excitable cells. There is as well the unavoidable disjunction between Ca2+ entry as measured by Ca2+ selective fluorescent dyes and electrophysiological methods. The patch clamp technique is extraordinarily powerful for examining the biophysical details of the function of an ion channel (Neher, E. and Sakmann, B. 1992. The patch clamp technique. Scientific American March:44-51), however the level of membrane control it both achieves and requires makes it less suited to identifying a channel's physiological role. Fluorescence techniques are very limited in obtaining biophysical detail but better able to study physiological roles. This disconnection makes it difficult to determine if the effects of physiologically relevant stimuli (as determined by fluorescence measurements) are reproduced at the electrophysiological level.

In accordance with the present invention it is anticipated that the mechanism of Ca2+ entry in electrically non-excitable cells involves a Ca2+ channel sharing characteristics with the T type family of voltage gated Ca2+ channels (Densmore, J. J., Haverstick, D. M., Szabo, G., and Gray, L. S. 1996. A voltage operable current is involved in activation-induced Ca2+ entry in human lymphocytes whereas I_{CRAC} has no apparent role. Am.J.Physiol. 271:C1494-C1503; Densmore, J. J., Szabo, G., and Gray, L. S. 1992. A voltage-gated calcium channel is linked to the antigen receptor in Jurkat T lymphocytes. FEBS Lett. 312:161-164; Haverstick, D. M. and Gray, L. S. Increased intracellular Ca2+ induces Ca2+ influx in human T lymphocytes. Molecular Biology of the Cell 4, 173-184. 1993; Haverstick, D. M., Densmore, J. J., and Gray, L. S. 1998. Calmodulin regulation of Ca2+ entry in Jurkat T cells. Cell Calcium 23:361-368). It could be argued that it is difficult to envision a physiologic role for voltage gated Ca2+channels in cells that do not have action potentials. This argument is, however, based upon the assumption that a voltage gated Ca2+ channel can be only be activated by an action potential. Such an assumption is false a priori because the means by which a protein can be regulated by imposed experimental conditions is not necessarily identical with, or even similar to, the mechanism by which it is controlled physiologically. Although secondary to regulation by membrane potential, the known biochemical regulation of voltage gated Ca2+ channels in a variety of systems (Heady, T. N., Gomora, J. C., Macdonald, T. L., and Perez-Reyes,E. 2001. Molecular pharmacology of T-type Ca2+ channels. Jpn.J.Pharmacol. 85:339-350; Hockerman, G. H., Peterson, B. Z., Johnson, B. D., and Catterall, W. A. 1997. Molecular determinants of drug binding and action on L-type calcium channels. Annu.Rev. Pharmacol. Toxicol. 37:361-396; Slish, D. F., Schultz, D., and Schwartz, A. 1992. Molecular biology of the calcium antagonist receptor. Hypertension 19:19-24) also suggests that the categorical distinction between electrical and biochemical regulation of Ca2+ channels may be somewhat simplistic.

We have taken an alternative approach to dissecting the Ca2+ entry pathway in electrically non-excitable cells. We first took advantage of Ca2+ entry blockade by Ni2+, as measured by fluorescence techniques (Merritt, J. E. and Rink, T. J. 1987. Regulation of cytosolic free calcium in fura-2-loaded rat parotid acinar cells. J.Biol.Chem. 262:17362-17369; Merritt, J. E., Jacob, R., and Hallam, T. J. 1989. Use of manganese to discriminate between calcium influx and mobilization from internal stores in stimulated human neutrophils. J.Biol.Chem. 264:1522-1527; Skryma, R., Mariot, P., Bourhis, X. L., Coppenolle, F. V., Shuba, Y., Abeele, F. V., Legrand, G., Humez, S., Boilly, B., and Prevarskaya, N. 2000. Store depletion and store-operated Ca2+ current in human prostate cancer LNCaP cells: involvement in apoptosis. J.Physiol.(Lond.) 527 Pt 1:71-83), to identify compounds in the published literature with a similar ability. The structure/activity relationship of these know compounds was used to guide the synthesis of novel compounds with enhanced potency to block Ca2+ entry into and proliferation of several cancer cell lines. Two representative novel compounds were then shown to block the Ca2+ current through the heterologously expressed α1H isoform of T type Ca2+ channels. Importantly, cell lines sensitive to the novel compounds express message for α1H, its δ25 splice variant, or both. These observations raise the possibility of directed chemical synthesis of compounds that inhibit Ca2+ entry and thereby proliferation of cancer cells.

In accordance with one embodiment a novel compound that inhibits Ca2+ entry, and thereby proliferation of cancer cells is provided. The compounds have the general structure:

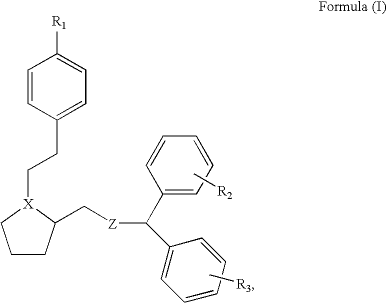

wherein

R₁ is selected from the group consisting of C₁-C₄ alkyl, hydroxy and C₁-C₄ alkoxy;

X is selected from the group consisting of N and CH;

Z is selected from the group consisting of NH, O, S and CH₂;

R₂ is selected from the group consisting of H, halo, NH₂, C₁-C₄ alky, hydroxy and C₁-C₄ alkoxy; and R₃ is selected from the group consisting of H, halo, NH₂, C₁-C₄ alkyl, hydroxy and C₁-C₄ alkoxy. In one embodiment R₁ is selected from the group consisting of C₁-C₄ alkyl, hydroxy and C₁-C₄ alkoxy, X is N, Z is O or CH₂, R₂ is H, halo, NH₂ or hydroxy and R₃ is H.

The novel compounds of the present invention can be combined with standard pharmaceutically acceptable carriers or other known anti-tumor and chemotherapeutic agents.

### Materials and Methods

### Synthesis of TH-1177:

TH-1177 was synthesized in three simple steps as described (Haverstick, D. M., Heady, T. N., Macdonald, T. L., and Gray, L. S. 2000. Inhibition of human prostate cancer proliferation in vitro and in a mouse model by a compound synthesized to block Ca2+ entry. Cancer Res 60:1002-1008). L-Proline methyl ester was coupled with 4-methoxyphenylacetic acid using benzotriazol-1-yl-oxytripyrrolidinephosphonium to generate methyl 1-[2-(4 methoxyphenyl)acetyl]pyrrolidine-2-carboxylate, a yellowish oil. The resulting amide was subsequently reduced to the amino alcohol with LiAlH₄ and AlCl₃ in tetrahydrofuran. The resulting colorless oil was coupled with 4-chlorobenzhydrol under Williamson conditions with catalytic p-toluenesulfonic acid in refluxing toluene. The final brownish oil was isolated by column chromatography, and its structure was confirmed by nuclear magnetic resonance and mass spectrometry. TH-1177 was dissolved in DMSO for use.

### Cell Lines and Maintenance:

Cancer cell lines were obtained from the American Type Culture Collection (Manassas, Va.). Cell lines were maintained in RPMI1640 supplemented with glutamine and 5% fetal bovine serum containing SerXtend (Irvine Scientific). The fetal bovine serum used for culture was heat-inactivated by maintaining the serum at 56° C. for 1 h.

### Measurement of the [Ca2+]i Concentration:

Cells were incubated in growth media containing 1 uM of the acetoxy-methyl ester of the Ca2+-sensitive fluorescent dye indo-1 (indo-1/AM; Molecular Probes, Eugene, Oreg.) for 1 h at 37° C. Cells were washed three times in buffer A [10 mM HEPES (pH 7.4),1 mM MgCl₂, 3 mM KCl, 1 mM CaCl₂, 140 mM NaCl, 0.1% glucose, and 1% fetal bovine serum] and suspended to a final concentration of 10⁶ cells/ml. Before stimulation, cells were warmed to 37° C. Changes in [Ca2+]i were monitored in an SLM 8100C. spectrofluorometer (SLM/Aminco; Urbana, Ill.) using previously published methods (Densmore, J. J., Haverstick, D. M., Szabo, G., and Gray, L. S. 1996. A voltage operable current is involved in activation-induced Ca2+ entry in human lymphocytes whereas I_{CRAC} has no apparent role. Am.J.Physiol. 271:C1494-C1503; Haverstick, D. M., Densmore, J. J., and Gray, L. S. 1998. Calmodulin regulation of Ca2+ entry in Jurkat T cells. Cell Calcium 23:361-368).

### Measurement of Cellular Proliferation:

LNCaP cells at 2.5×10⁴ cells/well or PC-3 cells at 5×10⁴ cells/well, both in a final volume of 100 I.JI, were plated in triplicate in standard flat-bottomed 96-well tissue culture plates in the presence of drug or vehicle (DMSO). Unless otherwise indicated, cells were grown for 48 h at 37° C. in a CO₂ incubator. Relative cell growth was determined with the CellTiter 96 aqueous cell proliferation assay (Promega, Madison, Wis.) as described by the manufacturer using an automated plate reader. Results were calculated in a blinded fashion and are the means of triplicate determinations.

### Results

Construction of a novel chemical library:

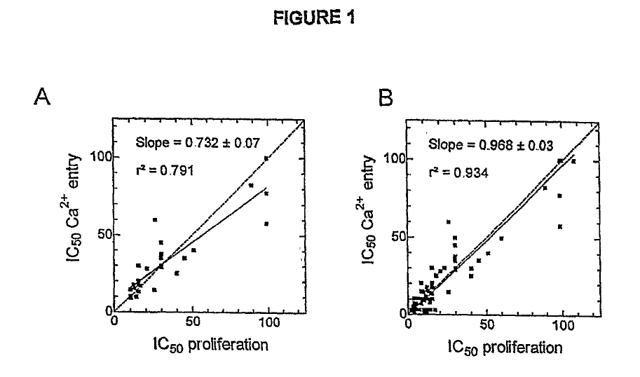

Extracellular Ni2+ blocks the Ca2+ entry pathway in electrically non-excitable cells (Merritt, J. E., Jacob, R., and Hallam, T. J. 1989. Use of manganese to discriminate between calcium influx and mobilization from internal stores in stimulated human neutrophils. J.Biol.Chem. 264:1522-1527; Jones, G. R. N. 1985. Cancer therapy: Phenothiazines in an unexpected role. Tumori 71:563-569) as well as the current through T type Ca2+ channels (Lee, J.-H., Gomora, J. C., Cribbs, L. L., and Perez-Reyes, E. 2000. Nickel block of three cloned T-type Ca channels: low concentrations selectively block α1H. Biophys.J 77:3042). We made use of these facts and conducted a search of the Medline database for compounds that block Ca2+ entry in any system that was also sensitive to inhibition of Ca2+ entry by Ni2+. The identified compounds were then used as the basis for a reiterated search. This strategy was continued until the only citations returned were those that had been retrieved already indicating that the database had been saturated. These agents, some of which are listed in Table 1, were tested for the ability to block proliferation of and Ca2+ entry into the Jurkat human cancer cell line. These compounds were tested in various cancer cell lines (Materials and Methods) with results similar to those obtained with the Jurkat cell line (data not shown). The correlation between these two inhibitory activities in the Jurkat cell line, expressed as IC₅₀'s, is shown in FIG. 1, panel A. The resulting structure-activity relationship (SAR) was used as a guide to synthesize novel chemical agents. These novel compounds exhibited enhanced inhibition of Ca2+ into and proliferation (Table 2). The slope of the regression line between the ability of the novel compounds to inhibit proliferation and block Ca2+ entry was 0.97 or very close to unity with an˜value of 0.93 (FIG. 1, panel B) compared to a slope of 0.73 (˜=0.79) for the known agents (FIG. 1, panel A). Because Ca2+ entry is required for proliferation (Berridge, M. J., Lipp, P., and Bootman, M. D. 2000. The versatility and universality of calcium signalling. Nat.Rev.Mol Cell Biol. 1:11-21), the slope of 0.97 should most appropriately be interpreted in a Bayesian fashion. This Bayesian analysis suggests that all of the effect of these compounds on proliferation is mediated through inhibition of Ca2+ entry.

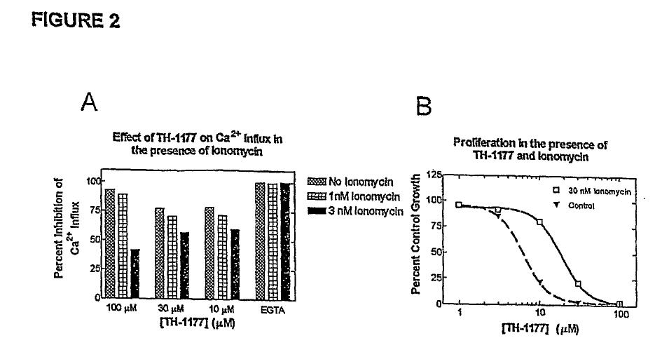

The Ca2+ ionophore ionomycin partially overcomes the effects of TH-1177. We have used one of our compounds, TH-1177, as the prototype for the others (Haverstick, D. M., Heady, T. N., Macdonald, T. L., and Gray, L. S. 2000. Inhibition of human prostate cancer proliferation in vitro and in a mouse model by a compound synthesized to block Ca2+ entry. Cancer Res 60:1002-1008). If TH-1177 is acting via inhibition of Ca2+ entry, its effects should be at least partially reversed by direct elevation of [Ca2+]i using a Ca2+ ionophore. As shown in FIG. 2, panel A, ionomycin overcame inhibition of Ca2+ entry by TH-1177 in a concentration dependent manner although there was no effect on proliferation of 30 nM ionomycin alone. Ionomycin also reduced the ability of TH-1177 to inhibit proliferation (FIG. 2, panel B) increasing the IC50 of TH-1177 from 4.6 uM in the presence of 30 uM ionomycin to 17.8 uM in its absence. This suggests that TH-1177 is acting to inhibit proliferation by inhibition of Ca2+ entry and is in accord with the relationship between Ca2+ entry and proliferation shown in FIG. 1.

Cancer cell lines sensitive to our agents express message for the α1H Ca2+ channel or its b25 splice variant.

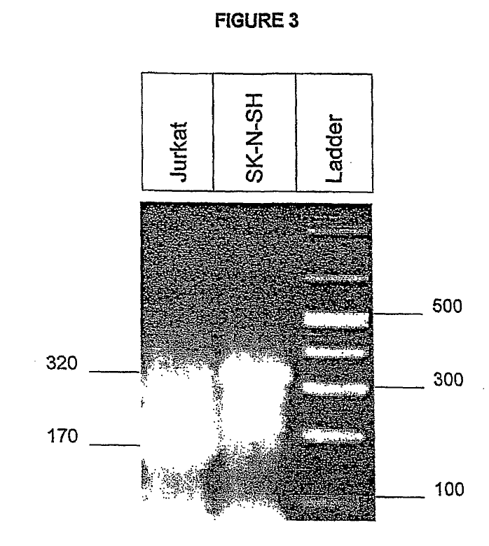

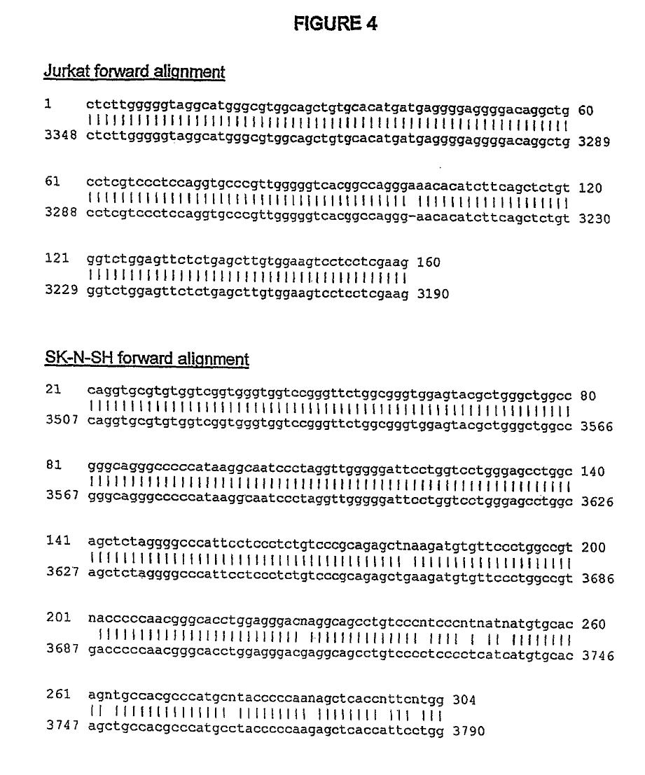

We have presented data previously suggesting that a member or members of the T type Ca2+ channel family have a role in mediating Ca2+ entry in electrically excitable cells (Densmore, J. J., Haverstick, D. M., Szabo, G., and Gray, L. S. 1996. A voltage operable current is involved in activation-induced Ca2+ entry in human lymphocytes whereas I_{CRAC} has no apparent role. Am.J.Physiol. 271:C1494-C1503; Densmore, J. J., Szabo, G., and Gray, L. S. 1992. A voltage-gated calcium channel is linked to the antigen receptor in Jurkat T lymphocytes. FEBS Lett. 312:161-164; Haverstick, D. M. and Gray, L. S. Increased intracellular Ca2+ induces Ca2+ influx in human T lymphocytes. Molecular Biology of the Cell 4, 173-184. 1993; Haverstick, D. M., Densmore, J. J., and Gray, L. S. 1998. Calmodulin regulation of Ca2+ entry in Jurkat T cells. Cell Calcium 23:361-368). It has been shown recently that a prostate cancer line expresses the α1H isoform of T type Ca2+ channels at levels that vary with differentiation status (Mariot, P., Vanoverberghe, K., Lalevee, N., Rossier, M. F., and Prevarskaya, N. 2002. Overexpression of an alpha 1H (Cav3.2) T-type calcium channel during neuroendocrine differentiation of human prostate cancer cells. J.Biol.Chem. 277:10824-10833). Using the same primers, we identified two different amplicons in cancer cell lines (FIG. 3). The 170 base amplicon, found in the malignant T cell line Jurkat, is similar to T type Ca2+ channel isoform α1H (UniGene cluster Hs.122359), with an expectation value of 3e-79 in both forward and reverse directions for Jurkat cells. For DU145 the expectation value in the forward direction was 1e-67 and in the reverse was 1e-70. The 320 base amplicon from the neuroblastoma cell line SK-N-SH is similar to the 825 splice variant (GenBank accession number AF223563), with an expectation value of 1e-141 in the reverse direction and 1e-135 in the forward direction. Also shown in FIG. 3 is the result of the RT-PCR assay using message obtained from the HL60 human leukemia cell line. The lack of a detectable PCR product is concordant with the resistance of this cell line to inhibition of proliferation or Ca2+ entry by our novel compounds (data not shown). The results of the Blast alignment of the two amplicons against the GenBank database are shown in FIG. 4. As shown in Table 3, several cancer cell lines express message for α1H, the 625 splice variant, or both. These observations suggest that the α1H and 625 products are candidates for mediating Ca2+ entry in at least some cancer cell lines and that expression of them is required for sensitivity to our novel chemical agents.

Diasteromers TH-1177 and TH-1211 inhibit proliferation of PC3 prostate cancer cells and block α1H with the same stereoselectively.

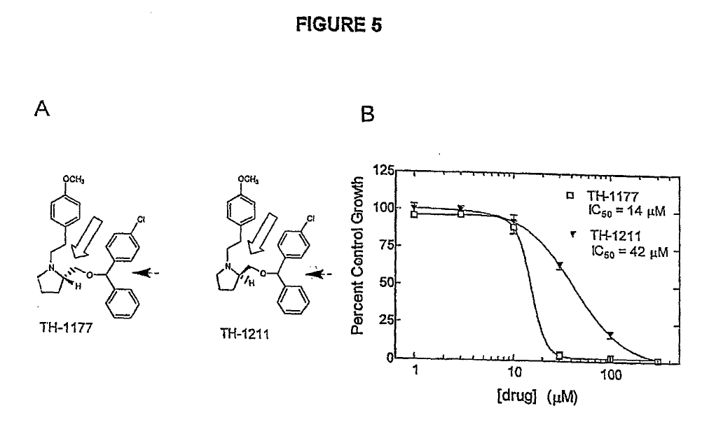

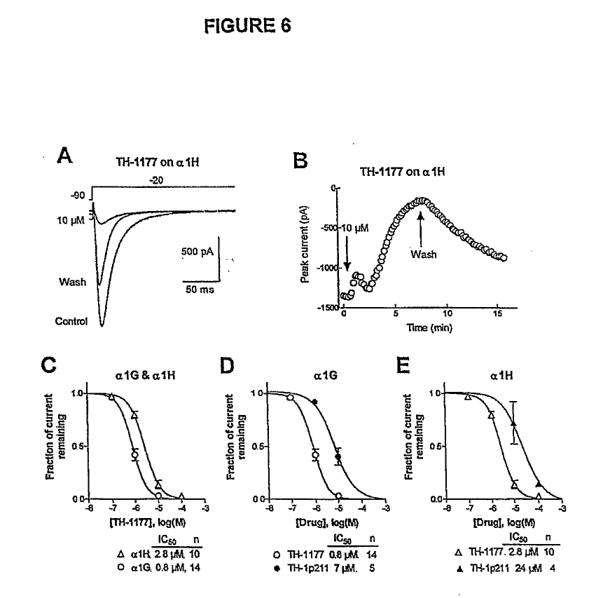

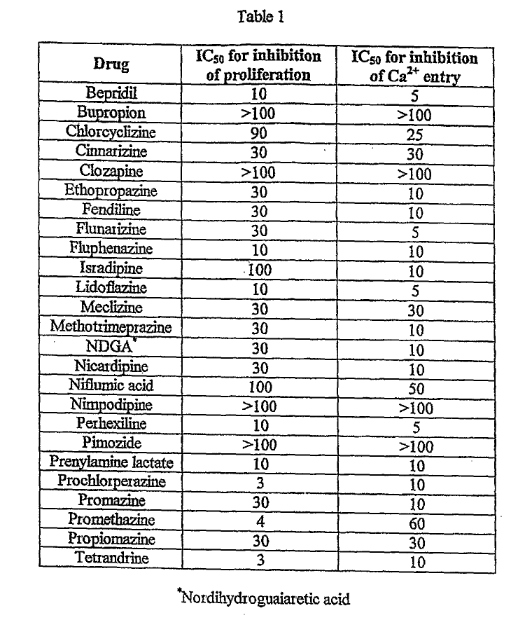

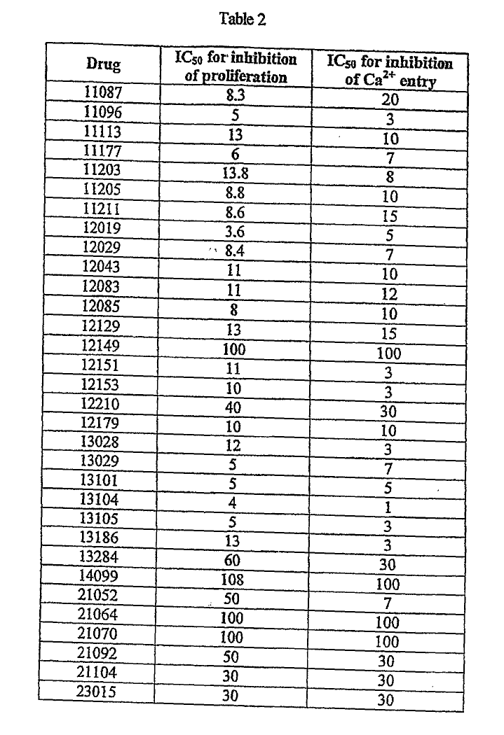

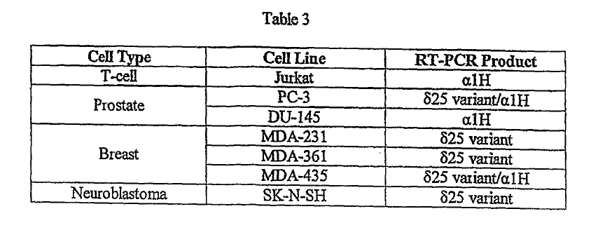

TH-1177 has two chiral centers and TH-1211 is its stereoisomer about one of them (FIG. 5, panel A). As shown in FIG. 5, panel B, TH-1177 is more potent at inhibiting proliferation of PC3 prostate cancer cells with an IC50 of 14 uM than is TH-1211 with an IC50 of 42 uM. TH-1177 and TH-1211 show the same rank order of potency at blocking the heterologously expressed, canonical α1H Ca2+ channel (FIG. 6). The IC50 for inhibition of transfected α1H by TH-1177 is 2.8 uM while the value for TH-1211 is 24 uM. Thus, when measured by Ca2+ selective fluorescent dyes, Ca2+ entry was similarly sensitive to TH-1177 and TH-1211 as was the Ca2+ current mediated by α1H. As importantly, each of these measures of Ca2+ influx showed the same relative difference in sensitivity to the stereoisomers. This shows the pharmacological correspondence between capacitative Ca2+ entry when measured by conventional means and Ca2+ entry mediated by α1H when measured by electrophysiological methods.

### Discussion

We have shown here the possibility that the α1H isoform of T type Ca2+ channels or its δ25 splice variant has a role in Ca2+ entry into and proliferation of electrically non-excitable cells. Our data show that novel compounds can be created based upon an SAR generated from compounds that are known to inhibit Ca2+ entry in systems that are also sensitive to Ni2+. Importantly, inhibition of proliferation of several cancer cell lines by these novel compounds is most likely via blockade of Ca2+ entry. The same cell lines that are sensitive to our agents express message for α1H Ca2+ channels, its δ25 splice variant, or both. The compounds were shown to inhibit the Ca2+ current mediated by α1H Ca2+ channels. TH-1177 and TH-1211 stereoselectively inhibit Ca2+ entry into and proliferation of cancer cell lines and show the same stereoselective block of canonical α1H. These data strongly suggest that the α1H Ca2+ channel and its 625 splice variant participate in Ca2+ entry in the cancer cell lines tested in these studies.

Linking biophysical analysis of Ca2+ channel function to a physiological function such as proliferation can pose challenges. We have demonstrated that our compounds block a heterologously expressed Ca2+ channel and that only those cancer cell lines with message for that channel, or its splice variant, are sensitive to inhibition by the same agents. Furthermore, TH-1177 is more potent at inhibiting Ca2+ entry via expressed α1H as measured by biophysical techniques than the stereoisomer of it, TH-1211. TH-1177 and TH-1211 also show the same rank order of potency at inhibiting proliferation and Ca2+ entry in cancer cell lines when these are assayed by more commonly used biochemical methods. The absolute potencies of the agents as measured by IC50 values are strikingly similar whether measured by biophysical or biochemical methods. Thus, the results from a combination of experimental approaches were synthesized into a picture of the likely mechanism of Ca+ entry in some cancer cells.

Expression of the α1H Ca2+ channel has been demonstrated in LNCaP cells and the expression level correlates with differentiation state (Mariot, P., Vanoverberghe, K., Lalevee, N., Rossier, M. F., and Prevarskaya, N. 2002. Overexpression of an alpha 1H (Cav3.2) T-type calcium channel during neuroendocrine differentiation of human prostate cancer cells. J.Biol.Chem. 277:10824-10833). Although the sequence of the 625 splice variant has been deposited in GenBank (accession number AF223563), its function has not been described to our knowledge. However, both are members of the T type Ca2+ channel family by sequence homology and have been assigned to the Hs.122359 UniGene cluster within the NCBI database. The physiological roles of T type Ca2+ channels are not wholly clear at present although they may playa role as pacemakers in the heart and central nervous system (Chemin, J., Monteil, A., Perez-Reyes, E., Bourinet, E., Nargeot, J., and Lory, P. 2002. Specific contribution of human T-type calcium channel isotypes (α_{1G}, α_{1H} and α_{1I}) to neuronal excitability. J.Physiol.(Lond.) 540:3-14; McDonald, T. F., Pelzer, S., Trautwein, W., and Pelzer, D. J. 1994. Regulation and modulation of calcium channels in cardiac, skeletal, and smooth muscle cells. Physiol Rev 74:365-507). The expression of these Ca2+ channels also appears to be developmentally regulated (Brooks, G., Harper, J. V., Bates, S. E., Haworth, R. S., Cribbs, L. L., Perez-Reyes,E., and Shattock, M. J. 1999. Over expression of the voltage-gated T-type calcium channel induces vascular smooth muscle cell proliferation. Circulation 100:1-209(Abstr.); Clozel, J. P., Ertel, E. A., and Ertel, S. I. 1999. Voltage-gated T-type Ca2+ channels and heart failure. Proc.Assoc.Am Physicians 111:429-437; Harper, J. V., McLatchie, L., Perez-Reyes,E., Cribbs, L. L., Shattock, M. J., and Brooks, G. 2000. T-type calcium channel expression is necessary for G1-S progression in vascular smooth muscle. Circulation 102:11-48 (Abstr.); Monteil, A., Chemin, J., Bourinet, E., Mennessier, G., Lory, P., and Nargeot, J. 2000. Molecular and functional properties of the human α_{1G} subunit that forms T-type calcium channels. J Biol.Chem. 275:6090-6100) and the data reported here suggest that both canonical α1H and its 625 splice variant are responsible for the Ca2+ entry required for proliferation of some cancer cell lines.

The presently described synthetic compounds may have clinical utility because treatment with TH-1177 of mice bearing xenografted human PC3 prostate cancer cells significantly extended the lifespan of them (Haverstick, D. M., Heady, T. N., Macdonald, T. L., and Gray, L. S. 2000. Inhibition of human prostate cancer proliferation in vitro and in a mouse model by a compound synthesized to block Ca2+ entry. Cancer Res 60:1002-1008). Thus, it is possible that Ca2+ channel entry inhibitors will provide clinicians with an addition to their armamentarium for the treatment of cancer. These results also raise the possibility that a reasonable and testable hypothesis for the mechanism of Ca2+ entry in electrically non-excitable cells has been overlooked. The observations presented here lay the groundwork for further developments in this area.

What is claimed is:

1. A method for treating a disease or condition in a mammal associated with influx of extracellular calcium via T type calcium channels, which comprises administering to the mammal a therapeutically effective amount of a T type calcium channel inhibitor, a prodrug thereof, or a pharmaceutically acceptable salt of said inhibitor or prodrug.

2. The method according to claim 1, wherein the disease or condition is selected from the group consisting of unstable angina, hypertension, epilepsy, neuropathic pain, petit mal seizure, absence seizure, age related macular degeneration, cancer, and pre-cancerous condition.

3. The method according to claim 1, wherein the T type calcium channel inhibitor blocks an α1H isoform of T type calcium channels or a δ25 splice variant thereof.

4. The method according to claim 1, wherein the T type calcium channel inhibitor has a structure represented by Formula (I): 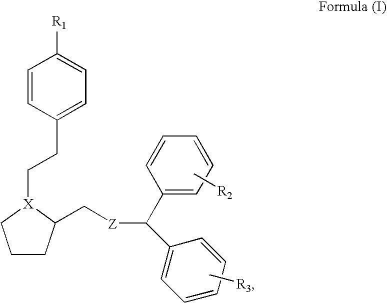
wherein
R₁ is selected from the group consisting of C₁-C₄ alkyl, hydroxy and C₁-C₄ alkoxy;
X is selected from the group consisting of N and CH;
Z is selected from the group consisting of NH, O, S and CH₂;
R₂ is selected from the group consisting of H, halo, NH₂, C₁-C₄ alkyl, hydroxy and C₁-C₄ alkoxy; and
R₃ is selected from the group consisting of H, halo, NH₂, C₁-C₄ alkyl, hydroxy and C₁-C₄ alkoxy. In one embodiment R₁ is selected from the group consisting of C₁-C₄ alkyl, hydroxy and C₁-C₄ alkoxy, X is N, Z is O or CH₂, R₂ is H, halo, NH₂ or hydroxy and R₃ is H.

5. A method for reducing proliferation of electrically non-excitable cells, which comprises administering a T type calcium channel inhibitor, wherein said T type calcium channels inhibitor blocks an α1H isoform of T type calcium channels or a δ25 splice variant thereof.

6. A method for inhibiting calcium entry into electrically non-excitable cells, which comprises administering a T type calcium channel inhibitor, wherein said T type calcium channels inhibitor blocks an α1H isoform of T type calcium channels or a δ25 splice variant thereof.

7. A pharmaceutical composition comprising a therapeutically effective amount of a compound of formula (I) as described in claim 4, a prodrug of said compound or a pharmaceutically acceptable salt of said compound or prodrug; and a pharmaceutically acceptable carrier, vehicle or diluent.

8. A method for the treatment of cancer or pre-cancerous condition in a mammal, which comprises administering to the mammal a therapeutically effective amount of a compound of formula (I) as described in claim 4 a prodrug thereof, or a pharmaceutically acceptable salt of said compound or prodrug in combination with one or more anti-tumor agent.

9. A pharmaceutical combination composition comprising a therapeutically effective amount of a combination of a compound of formula (I) as described in claim 4, a prodrug thereof, or a pharmaceutically acceptable salt of said compound or prodrug; and one or more anti-tumor agent.
